(19) United States
(12) Patent Application Publication
(10) Pub. No.: US 2007/0073178 A1
(43) Pub. Date: Mar. 29, 2007

(54) Monitoring device for measuring calorie expenditure

(75) Inventors: Ray Browning, Denver, CO (US); Christopher Hall, San Francisco, CA (US); Matthew Banet, Del Mar, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Berkeley HeartLab, Inc.

(21) Appl. No.: 11/522,565

(22) Filed: Sep. 18, 2006

Related U.S. Application Data
(60) Provisional application No. 60/721,665, filed on Sep. 29, 2005.

(51) Int. Cl. A61B 5/04

(52) U.S. Cl. 600/519

(57) ABSTRACT

The invention provides a monitoring device that features: 1) a cardiac sensor component with at least one light-emitting diode and a photodetector; 2) a pedometer component with at least one motion-sensing component (e.g., an accelerometer); and 3) a wireless component with a wireless interface that communicates with an external weight scale. The device also features a microprocessor in electrical communication with the cardiac sensor, pedometer, and wireless components and configured to analyze: 1) a signal from the cardiac sensor component to generate heart rate information; 2) a signal from the pedometer component to generate exercise information; 3) heart rate and exercise information to generate calorie information; and 4) a signal from the external weight scale to calculate weight information (e.g., weight and percent body fat).

### CROSS REFERENCES TO RELATED APPLICATION

This application claims the benefit of U.S. Provisional Application Ser. No. 60/721,665 filed on Sep. 29, 2005 and is hereby incorporated by reference.

### BACKGROUND OF THE INVENTION

1. Field of the Invention

The present invention relates to medical devices for monitoring information, such as heart rate and calories burned, from a subject.

2. Description of the Related Art

Pedometers are common devices that typically include a motion-sensitive component, such as an accelerometer or a tilt switch, that typically generates an analog voltage that peaks in response to motion (e.g., steps). A microcontroller can receive the analog voltage, digitize it, and then process it by counting the peaks to determine a subject's steps. Heart rate monitors are also common devices that measure a subject's heart rate, typically by measuring a biometric signal (i.e., by processing an electrical signal collected by an electrode, such as that used in an ECG) or an optical plethysmograph (i.e., by processing an optical signal collected by a pulse oximeter).

Pulse oximeters are typically worn on a patient's finger or ear lobe, and feature a processing module that analyzes data generated by an optical module. The optical module typically includes first and second light sources (e.g., light-emitting diodes, or LEDs) that transmit optical radiation at, respectively, red (λ˜630-670 nm) and infrared (λ˜800-1200nm) wavelengths. The optical module also features a photodetector that detects radiation transmitted or reflected by an underlying artery. Typically the red and infrared LEDs sequentially emit radiation that is partially absorbed by blood flowing in the artery. The photodetector is synchronized with the LEDs to detect transmitted or reflected radiation. In response, the photodetector generates a separate radiation-induced signal for each wavelength. The signal, called a plethysmograph, is an optical waveform that varies in a time-dependent manner as each heartbeat varies the volume of arterial blood, and hence the amount of transmitted or reflected radiation. A microprocessor in the pulse oximeter processes the relative absorption of red and infrared radiation to determine the oxygen saturation in the patient's blood. A number between 94%-100% is considered normal, while a value below 85% typically indicates the patient requires hospitalization.

### SUMMARY OF THE INVENTION

In one aspect the invention provides a monitoring device that features: 1) a cardiac sensor component with at least one LED and a photodetector; 2) a pedometer component with at least one motion-sensing component (e.g., an accelerometer); and 3) a wireless component with a wireless interface that communicates with an external weight scale. The device also features a microprocessor in electrical communication with the cardiac sensor, pedometer, and wireless components and configured to analyze: 1) a signal from the cardiac sensor component to generate heart rate information; 2) a signal from the pedometer component to generate exercise information; 3) heart rate and exercise information to generate calorie information; and 4) a signal from the external weight scale to calculate weight information (e.g., weight and percent body fat). The monitoring device also includes a transmitting component (e.g. a serial port or wireless interface) that transmits the heart rate, exercise, calorie, and weight information to an external device, such as a personal computer connected to the Internet.

In embodiments, the microprocessor is configured to operate a computer algorithm that processes the heart rate and exercise information to generate calorie information, such as calories burned. For example, the algorithm can process the physical activity information to determine whether a subject is at rest or undergoing exercise, and once this is determined compare the heart rate information to pre-determined calibration information to determine an amount of calories burned by the subject. More specifically, the calibration information can include a predetermined data table or mathematical function that correlates oxygen consumed as a function of heart rate. The algorithm can then calculate caloric expenditure from the amount of oxygen consumed.

The invention has many advantages, particularly in providing a small-scale, low-cost device that rapidly measures health-related indicators such as blood pressure, heart rate, and blood oxygen content. In embodiments, the device makes blood pressure measurements without using a cuff in a matter of seconds, meaning patients can easily monitoring device this property with minimal discomfort. In this way the monitoring device combines all the benefits of conventional blood-pressure measuring devices without any of the obvious drawbacks (e.g., restrictive, uncomfortable cuffs). Its measurement, made with an optical ‘pad sensor’, is basically unobtrusive to the patient, and thus alleviates conditions, such as a poorly fitting cuff, that can erroneously affect a blood-pressure measurement. Ultimately this allows patients to measure their vital signs throughout the day (e.g., while at work), thereby generating a complete set of information, rather than just a single, isolated measurement. Physicians can use this information to diagnose a wide variety of conditions, particularly hypertension and its many related diseases.

The device additionally includes a simple wired or wireless interface that sends vital-sign information to a personal computer. For example, the device can include a Universal Serial Bus (USB) connector that connects to the computer's back panel. Once a measurement is made, the device stores it on an on-board memory and then sends the information through the USB port to a software program running on the computer. Alternatively, the device can include a short-range radio interface (based on, e.g., Bluetooth™ or 802.15.4) that wirelessly sends the information to a matched short-range radio within the computer. The software program running on the computer then analyzes the information to generate statistics on a patient's vital signs (e.g., average values, standard deviation, beat-to-beat variations) that are not available with conventional devices that make only isolated measurements. The computer can then send the information through a wired or wireless connection to a central computer system connected to the Internet.

The central computer system can further analyze the information, e.g. display it on an Internet-accessible website. This means medical professionals can characterize a patient's real-time vital signs during their day-to-day activities, rather than rely on an isolated measurement during a medical check-up. The website typically features one or more web pages that display the blood test, vital sign, exercise, and personal information. In embodiments, the website includes a first web interface that displays information for a single patient, and a second web interface that displays information for a group of patients. For example, a medical professional (e.g. a physician, nurse, nurse practitioner, dietician, or clinical educator) associated with a group of patients could use the second web interface to drive compliance for a disease-management program. Both web interfaces typically include multiple web pages that, in turn, feature both static and dynamic content, described in detail below.

The website can also include a messaging engine that processes real-time information collected from the device to, among other things, help a patient comply with a disease-management program, such as a personalized cardiovascular risk reduction program. The messaging engine analyses blood test, vital sign, exercise, and personal information, taken alone or combined, to generate personalized, patient-specific messages. Ultimately the Internet-based system, monitoring device, and messaging engine combine to form an interconnected, easy-to-use tool that can engage the patient in a disease-management program, encourage follow-on medical appointments, and build patient compliance. These factors, in turn, can help the patient lower their risk for certain medical conditions.

These and other advantages of the invention will be apparent from the following detailed description and from the claims.

### BRIEF DESCRIPTION OF THE DRAWINGS

FIG. 1A is a semi-schematic view of a portable, small-scale monitoring device that measures blood pressure, pulse oximetry, heart rate, glucose levels, weight, steps traveled, and calories burned;

FIG. 1B is a semi-schematic view of the monitoring device of FIG. 1A worn on a patient's belt;

FIG. 2 is a schematic view of an Internet-based system that receives information from the monitoring device of FIGS. 1A and 1B through a wired connection;

FIG. 3 is a schematic diagram of the electrical components of the monitoring devices of FIGS. 1A and 1B;

FIG. 4a is a flow chart describing a first algorithm used by the monitoring devices of FIGS. 1A and 1B to calculate calories burned;

FIG. 4b is a flow chart describing a second algorithm used by the monitoring devices of FIGS. 1A and 1B to calculate calories burned; and

FIG. 5 is a flow chart describing a second algorithm used by the monitoring devices of FIGS. 1A and 1B to calculate calories burned.

### DETAILED DESCRIPTION OF THE INVENTION

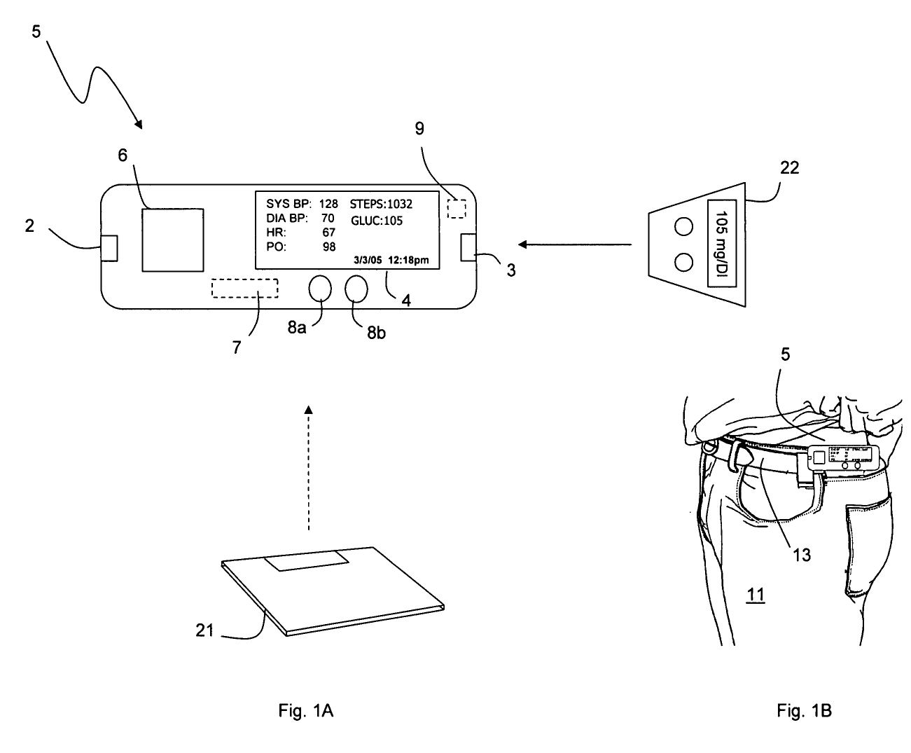

FIGS. 1A and 1B show a portable, small-scale monitoring device 5 that measures information such as blood pressure, pulse oximetry, heart rate, glucose levels, calories burned and steps traveled from a patient 1. The monitoring device 5, typically worn on the patient's belt 13, features: i) an integrated, optical ‘pad sensor’ 6 that cufflessly measures blood pressure, pulse oximetry, and heart rate from a patient's finger as described in more detail below; and ii) an integrated pedometer circuit 9 that measures steps and, using one or more algorithms, calories burned. To receive information from external devices, the monitoring device 5 also includes: i) a serial connector 3 that connects and downloads information from an external glucometer 22; and ii) a short-range wireless transceiver 7 that receives information such as body weight and percentage of body fat from an external scale 21. The patient views information from a liquid crystal display (LCD) display 4 mounted on the monitoring device 5, and can interact with the monitoring device 5 (e.g., reset or reprogram it) using a series of buttons 8a, 8b.

The monitoring device can be used for a variety of applications relating to, e.g., disease management, health maintenance, and medical diagnosis.

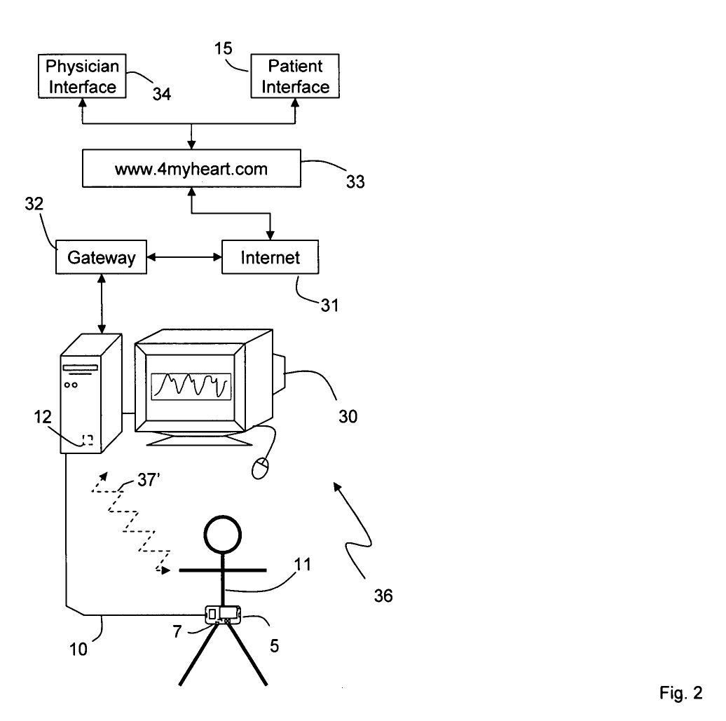

FIG. 2 shows a preferred embodiment of an Internet-based system 36 that operates in concert with the small-scale monitoring device 5 to send information from the patient 11 to an Internet-accessible website 33. There, a user can access the information using a conventional web browser through a patient interface 15 or a physician interface 34. Typically the patient interface 15 shows information from a single user, whereas the physician interface 34 displays information for multiple patients. In both cases, information flows from the monitoring device 5 through a USB cable 10 to an external device, e.g., a personal computer 30. The personal computer 30 connects to the Internet 31 through a wired gateway software system 32, such as an Internet Service Provider.

In other embodiments, the small-scale monitoring device 5 transmits patient information using a short-range wireless transceiver 7 through a short-range wireless connection 37 (e.g., Bluetooth™, 802.15.4, part-15) to the personal computer 30. For example, the small-scale monitoring device 5 can transmit to a matched transceiver 12 within (or connected to) the personal computer 30.

During typical operation, the patient 11 uses the monitoring device 5 for a period of time ranging from a 1-3 months. Typically the patient 11 takes measurements a few times throughout the day, and then uploads the information to the Internet-based system 36 using a wired connection. Alternatively, the monitoring device 5 can measure the patient 11 continuously during periods of exercise. To view patient information sent from the monitoring device 5, the patient 11 (or other user) accesses the appropriate user interface hosted on the website 33 through the Internet 31.

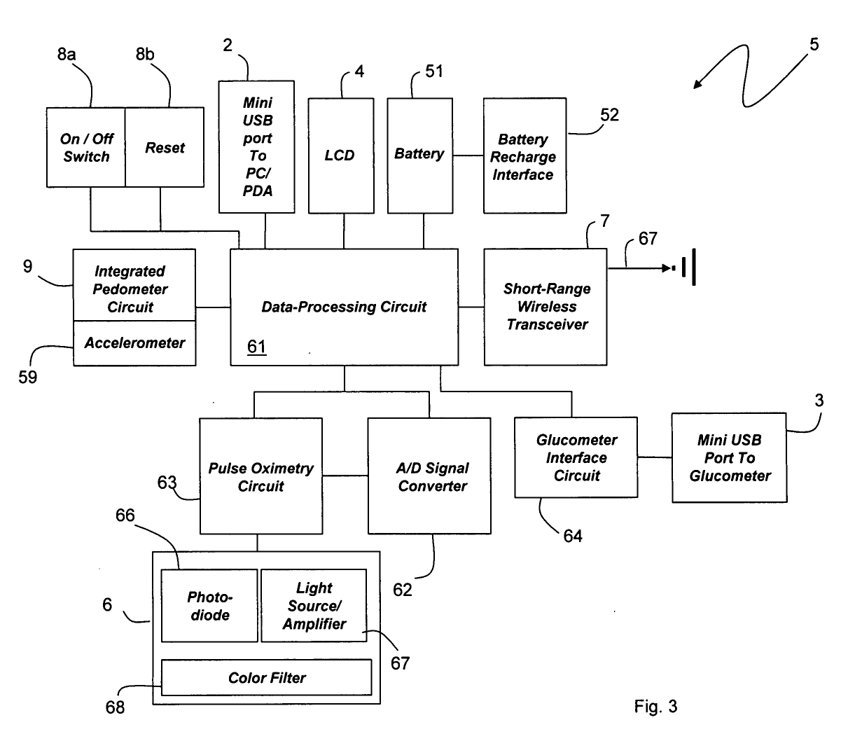

FIG. 3 shows a preferred embodiment of the electronic components within the monitoring device 5. A data-processing circuit 61 controls: i) a pulse oximetry circuit 63 connected to an optical pad sensor 6; ii) LCD 4; iii) a glucometer interface circuit 64 that connects to an external glucometer through a mini USB port 3; iv) an integrated pedometer circuit 9 featuring an accelerometer 59; and v) a short-range wireless transceiver 7. During operation, the optical pad sensor 6 generates an optical waveform that the data-processing circuit 61 processes to measure blood pressure, pulse oximetry, and heart rate as described in more detail below. The sensor 6 combines a photodiode 66, color filter 68, and light source/amplifier 67 on a single silicon-based chip. The light source/amplifier 67 typically includes light-emitting diodes that generate both red (λ˜600 nm) and infrared (λ˜940 nm) radiation. As the heart pumps blood through the patient's finger, blood cells absorb and transmit varying amounts of the red and infrared radiation depending on how much oxygen binds to the cells′ hemoglobin. The photodiode 66 detects transmission at both red and infrared wavelengths, and in response generates a radiation-induced current that travels through the sensor 6 to the pulse-oximetry circuit 63. The pulse-oximetry circuit 63 connects to an analog-to-digital signal converter 62, which converts the radiation-induced current into a time-dependent optical waveform. The analog-to-digital signal converter 62 sends the optical waveform to the data-processing circuit 61 that processes it to determine blood pressure, pulse-oximetry, and heart rate, which are then displayed on the LCD 4. Once information is collected, the monitoring device 5 can send it through a mini USB port 2 to a personal computer 30 as described with reference to FIG. 2.

In other embodiments, the monitoring device 5 connects through the mini USB port 3 and glucometer interface circuit 64 to an external glucometer to download blood-glucose levels. The monitoring device 5 also processes information from an integrated pedometer circuit 9 to measure steps and amount of calories burned, as described below.

The monitoring device 5 includes a short-range wireless transceiver 7 that sends information through an antenna 67 to a matched transceiver embedded in an external device, e.g. a personal computer. The short-range wireless transceiver 7 can also receive information, such as weight and body-fat percentage, from an external scale. A battery 51 powers all the electrical components within the small-scale monitoring device 5, and is preferably a metal hydride battery (generating 3-7V) that can be recharged through a battery-recharge interface 2. The battery-recharge interface 52 can receive power through a serial port, e.g. a computer's USB port. Buttons control functions within the monitoring device such as an on/off switch 8a and a system reset 8b.

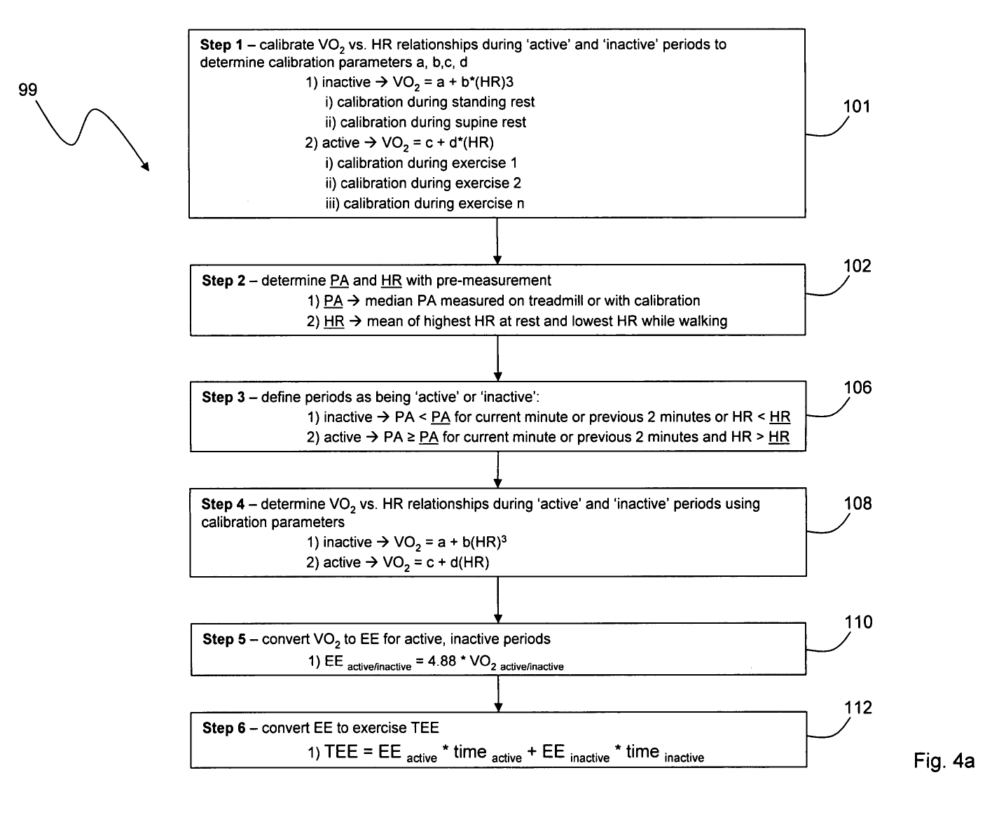

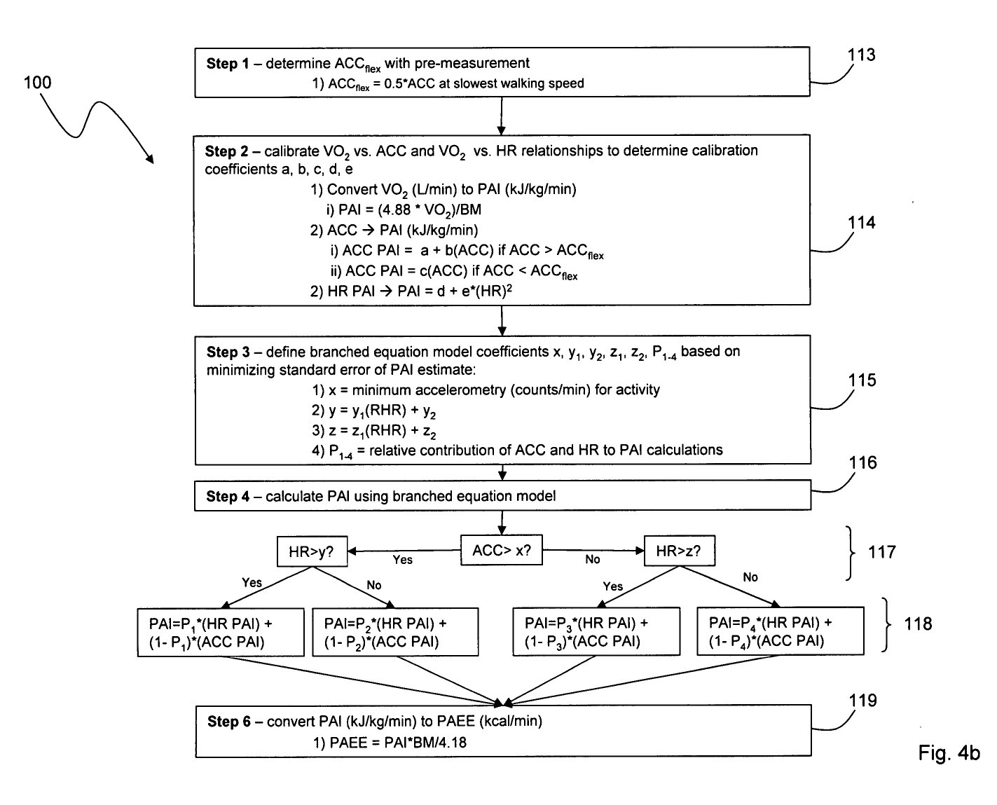

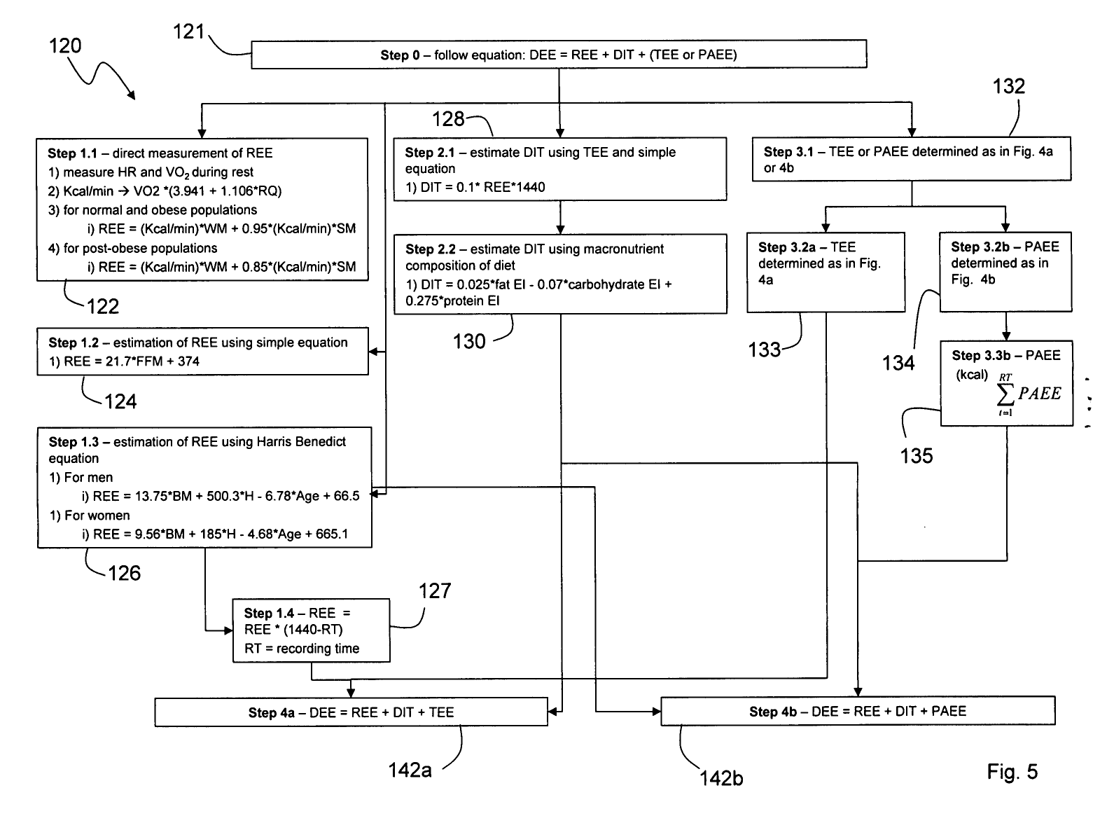

FIG. 4a shows a flow chart describing an algorithm 100 used by the monitoring device of FIGS. 1A and 1B to calculate an amount of calories burned during active and inactive periods. Parameters used in this calculation are defined in Table 1, below.

TABLE 1
Parameter Definitions

| | |
|---|---|
| PA - | physical activity level measured with accelerometer |
| | (counts/minute) |
| PAI - | physical activity (kJ/kg/minute) |
| PA - | PA threshold; median PA measured on treadmill or with |
| | calibration (counts/minute) |
| PA_{flex} - | physical activity flex point; 50% of mean PA |
| | (counts/minute) |
| HR - | heart rate measured with heart rate monitor |
| | (beats/minute) |
| HR - | HR threshold; mean of highest HR at rest and lowest HR |
| | while walking (beats/minute) |
| VO₂ - | oxygen consumption (liters/minute) |
| EE - | acute energy expenditure (kcal/minute) |
| DEE - | direct energy expenditure (kcal) |
| TEE - | total energy expenditure (kcal) |
| REE - | resting energy expenditure (kcal/day) |
| PAEE - | physical activity energy expenditure (kJ/kg/minute) |
| ACC - | accelerometer output (counts/min) |
| DIT - | dietary induced thermogenesis (kJ) |
| FFM - | fat free mass (kg) |
| EI - | energy intake (kJ) |
| BM - | body mass (kg) |
| H - | height (m) |
| Age - | age (years) |
| WM - | minutes awake each day (minutes/day) |
| SM - | minutes sleeping each day (minutes/day) |
| RT - | recording time (the number of minutes the device is on) |

The algorithm 100, which uses a patient's physical activity (PA) level and heart rate (HR), is based on a methodology developed by Moon and Butte (Moon J K and Butte N F; Combined heart rate and activity levels improve estimates of oxygen consumption and carbon dioxide production rates; J appl Physiol 81: 1754-1761, 1996), the contents of which are incorporated herein by reference.

As a first step 101, the algorithm 99 features a process that calibrates the monitoring device so that it can accurately measure calories burned during exercise. During the first step 101 VO₂ and HR are simultaneously measured during simulated, representative ‘active’ and ‘inactive’ periods, defined below. For example, VO₂ can be measured using indirect calorimetry while HR is measured using any number of techniques (e.g., ECG). VO₂ is then plotted as a function of HR for both the active and inactive periods. The resultant data are then fit with either a quadratic equation (for the inactive periods) or a linear equation (for the active periods), show below, to yield calibration parameters a, b, c, d. These calibration parameters will be most accurate if they are measured from a population that is representative to patients actually using the device.
inactive
VO₂=a+b*(HR)³
active
VO₂=c+d*(HR)

Typically the calibration process lasts a few hours and data describing VO₂ and HR are collected every minute. Active and inactive periods for the calibration process typically include the following:
inactive
1. 30 minutes of supine rest
2. 15 minutes of standing rest
active
1. 36 minutes of simulated daily activities
   a. level walking at 2 mph for 6 minutes
   b. level walking at 4 mph for 6 minutes
   c. level jogging at 6 mph for 6 minutes
   d. gardening or lawn care (mowing, raking, shoveling) for 6 minutes
   e. household chores (vacuuming, sweeping and stacking groceries) for 6 minutes
      Once calibrated, the algorithm 99 includes a second step 102 that determines threshold values for both PA (defined as PA) and HR (defined as HR). PA is typically the median value of PA determined while the patient is on the treadmill during the first step 101. HR is typically the mean of highest HR measured at rest and the lowest measured HR during walking. Using the threshold values, the algorithm 99 includes a third step 106 that measures data from the subject to define periods as being either ‘active’ or ‘inactive’. For example, the subject is determined to be in an inactive state if PA<PA for one or more minutes, or alternatively if HR<HR. Alternatively, the subject is determined to be in an active state if PA≧PA for at least one minute and if HR>HR. Using the calibration parameters a, b, c, d determined from calibration during the first step 101, and the subject's active or passive state determined during the third step 106, the algorithm then calculates the subject's oxygen consumed (VO₂) during a fourth step 108. Specifically, the algorithm records HR during active or inactive periods, and then using the calibration parameters calculates VO₂ using either the above-mentioned quadratic equation (for an inactive period) or linear equation (for an active period). During a fifth step 110 the algorithm 100 converts VO₂ to acute energy expenditure (EE) for both active and inactive periods using the equation:
      EE_{active/inactive}=4.88 *VO_{2, active/inactive}
      During a sixth step 112 the algorithm converts EE (with units of kcal/minute) to total energy expenditure (TEE) using the total amount of time of either the active or inactive period. The time is typically measured in one-minute increments with a real-time clock within the monitoring device:
      TEE=EE_{active}*time_{active}+EE_{inactive}*time_{inactive}
      The sixth step 110 yields the amount of calories burned by the subject.

FIG. 4b shows an alternate embodiment of the algorithm 99 shown in FIG. 4a used to calculate PAEE. The figure shows a flow chart illustrating an algorithm 100 that features a first step 113 where a parameter related to accelerometer output called ACC_{flex}is determined from ACC (in counts/minute). During a second step 114 the algorithm calibrates VO₂ vs. ACC and VO₂ vs. HR relationships to determine the calibration coefficients a, b, c, d, e. As with FIG. 4a, these calibration parameters will be most accurate if they are measured from a population that is representative to patients actually using the device. During a third step 115, after the calibration parameters are determined, the algorithm 100 defines branched equation model coefficients x, Y₁, Y₂, Z₁, Z₂, P₁₋₄ based on minimizing standard error of PAI estimate. During a fourth step 116 the algorithm calculates PAI using a series of branched equations 117, 118, using the coefficients from the third step 115. This leads to a fifth step 118 wherein the algorithm converts PAI (with units of kJ/kg/min) to PAEE (kcal/min).

The branched equations are defined in more detail in the following reference, the contents of which are incorporated herein by reference: Brage S, Brage N, Franks P W, Ekelund U, Wong M, Andersen L B, Froberg K, and Wareham N J; Branched equation modeling of simultaneous accelerometry and heart rate monitoring improves estimate of directly measured physical activity energy expenditure; J appl Physiol 96: 343-351, 2004. The branched equations process values of HR and PA by comparing them with benchmark values, and in response assign percentages that define the relative contribution of these parameters to PAEE. These percentages will vary depending on the group used for the calibration process, and ultimately determine the total value for PAEE.

FIG. 5 shows a flow chart illustrating a second algorithm 120 used within the device to calculate the amount of calories a subject burns during both active and inactive periods. The algorithm 120 can use one of three possible steps 122, 124, 126 to calculate REE. For example, during a first step 122 REE is measured directly by first using a calibration step that determines HR and VO₂ during rest; this method is similar to that used for the first step 101 for the algorithm 100 described with reference to FIG. 4. VO₂ can be measured as described in steps 1-4 of the algorithm 100, and REE is calculated with the following equations:
Kcal/min→VO₂*(3.941+1.106*RQ)
for normal and obese populations
   REE=(Kcal/min)*WM+0.95*(Kcal/min)*SM
for post-obese populations
   REE=(Kcal/min)*WM+0.85*(Kcal/min)*SM
   Using an alternate first step 124 REE is determined using simple equation that takes into account the patient's fat-free mass (FFM):
   REE=21.7*FFM+374
   In this case, FFM is the patient's mass not attributed to fat, and is typically measured directly or calculated from a patient's body-mass index.

In another alternative first step 126 estimates REE using the Harris-Benedict equation:
for men
   REE=13.75*BM+500.3*H−6.78*Age+66.5
for women
   REE=9.56*BM+185*H−4.68*Age+665.1

In yet another alternate first step 127, REE calculated as described above is modified using recording time (RT), i.e.:
REE′=REE*(1440 −RT)

Once REE is determined, the algorithm 120 uses a second step 128 to estimate DIT using TEE and the equation:
DIT=0.1*TEE

Alternatively, DIT is calculated by estimating the macronutrient composition of the subject's diet. This is done using the following equation for the second step 130 of the algorithm 120:
DIT=0.025*fatEl −0.07*carbohydrateEl+0.275*proteinEl
During a third step 132 the algorithm uses TEE (described above with reference to FIG. 4a) or PAEE (described above with reference to FIG. 4b). For example, in one part of the third step 133, TEE is determined as described above, and then combined with the first and second steps to determine DEE 142a. In an alternate step 134, PAEE is determined using calibration information that describes the relationship between both PA and HR and VO₂ as described above. Once REE (step 1), DIT (step 2), PAEE (step 3) or TEE (step 3) are determined, the algorithm 120 uses a fourth step 142a,b to determine DEE:
DEE=REE+DIT+PAEE
or
DEE=REE+DIT+TEE

Methods for processing optical and electrical waveforms to determine blood pressure without using a cuff are described in the following co-pending patent applications, the entire contents of which are incorporated by reference: 1) CUFFLESS BLOOD-PRESSURE MONITORING DEVICE AND ACCOMPANYING WIRELESS, INTERNET-BASED SYSTEM (U.S. Ser. No 10/709,015; filed Apr. 7, 2004); 2) CUFFLESS SYSTEM FOR MEASURING BLOOD PRESSURE (U.S. Ser. No. 10/709,014; filed Apr. 7, 2004); 3) CUFFLESS BLOOD PRESSURE MONITORING DEVICE AND ACCOMPANYING WEB SERVICES INTERFACE (U.S. Ser. No. 10/810,237; filed Mar. 26, 2004); 4) VITAL-SIGN MONITORING DEVICE FOR ATHLETIC APPLICATIONS (U.S. Ser. No.; filed Sep. 13, 2004); 5) CUFFLESS BLOOD PRESSURE MONITORING DEVICE AND ACCOMPANYING WIRELESS MOBILE DEVICE (U.S. Ser. No. 10/967,511; filed Oct. 18, 2004); and 6) BLOOD PRESSURE MONITORING DEVICEING DEVICE FEATURING A CALIBRATION-BASED ANALYSIS (U.S. Ser. No. 10/967,610; filed Oct. 18, 2004); 7) PERSONAL COMPUTER-BASED VITAL SIGN MONITORING DEVICE (U.S. Ser. No. 10/906,342; filed Feb. 15, 2005); and 8) PATCH SENSOR FOR MEASURING BLOOD PRESSURE WITHOUT A CUFF (U.S. Ser. No. 10/906,315; filed Feb. 14, 2005).

Still other embodiments are within the scope of the following claims.

I claim as my invention:

1. A monitoring device comprising:
a cardiac sensor component comprising at least one light-emitting diode and a photodetector;
a pedometer component comprising at least one motion-sensing component;
a wireless component comprising a wireless interface configured to communicate with an external weight scale;
a microprocessor in electrical communication with the cardiac sensor, pedometer, and wireless components and configured to analyze: i) a signal from the cardiac sensor component to generate heart rate information; ii) a signal from the pedometer component to generate exercise information; iii) heart rate and exercise information to generate calorie information; and iv) a signal from the external weight scale to calculate weight information; and
a transmitting component for transmitting the heart rate, exercise, calorie, and weight information to an external device.

2. The monitoring device of claim 1, wherein the microprocessor is configured to operate a computer algorithm that processes the heart rate and exercise information to generate calorie information.

3. The monitoring device of claim 2, wherein the algorithm is further configured to process the physical activity information to determine whether a subject is at rest or undergoing exercise.

4. The monitoring device of claim 3, wherein the algorithm is further configured to compare the heart rate information to pre-determined calibration information to determine an amount of calories burned by the subject.

5. The monitoring device of claim 4, wherein the calibration information comprises a data table that correlates oxygen consumed as a function of heart rate.

6. The monitoring device of claim 5, wherein the algorithm is further configured to calculate caloric expenditure from an amount of oxygen consumed.

7. The monitoring device of claim 1, wherein the motion-sensing device is an accelerometer.

8. The monitoring device according to claim 1, wherein the transmitting component is a serial connection.

9. The monitoring device according to claim 8, wherein the serial connection is a USB connection.

10. The monitoring device according to claim 1, wherein the transmitting component is a wireless transceiver that operates a wireless protocol.

11. The monitoring device according to claim 10, wherein the wireless protocol is based on Bluetooth™, 802.11a, 802.11b, 802.1g, or 802.15.4.

12. The monitoring device according to claim 1, wherein the weight information is weight and percentage body fat.

13. The monitoring device according to claim 1, wherein the external device that receives the heart rate, exercise, calorie, and weight information is a personal computer.

14. The monitoring device according to claim 1, wherein the personal computer comprises a software component that collects the heart rate, exercise, calorie, and weight information and transmits this information to an Internet-accessible website.

15. A monitoring device comprising:
a cardiac sensor component comprising at least one light-emitting diode and a photodetector;
a pedometer component comprising at least one motion-sensing component;
a wireless component comprising a wireless interface configured to communicate with an external weight scale;
a microprocessor in electrical communication with the cardiac sensor, pedometer, and wireless components and configured to operate a computer program that:
1) analyzes: i) a signal from the cardiac sensor component to generate heart rate information; ii) a signal from the pedometer component to generate exercise information; and iii) a signal from the external weight scale to calculate weight information; and 2) analyzes: i) exercise information to determine whether a subject is at rest or undergoing exercise; and ii) heart rate information in combination with a pre-determined calibration information to determine an amount of calories burned by the subject; and
a transmitting component for transmitting the heart rate, exercise, calorie, and weight information to an external device.

16. The monitoring device according to claim 15, wherein the external device that receives the heart rate, exercise, calorie, and weight information is a personal computer.

17. The monitoring device according to claim 16, wherein the personal computer comprises a software component that collects the heart rate, exercise, calorie, and weight information and transmits this information to an Internet-accessible website.

18. A system comprising:
a monitoring device comprising:
a cardiac sensor component comprising at least one light-emitting diode and a photodetector;
a pedometer component comprising at least one motion-sensing component;
a wireless component comprising a wireless interface configured to communicate with an external weight scale;
a microprocessor in electrical communication with the cardiac sensor, pedometer, and wireless components and configured to operate a computer program that:
1) analyzes: i) a signal from the cardiac sensor component to generate heart rate information; ii) a signal from the pedometer component to generate exercise information; and iii) a signal from the external weight scale to calculate weight information; and 2) analyzes: i) exercise information to determine whether a subject is at rest or undergoing exercise; and ii) heart rate information in combination with a pre-determined calibration information to determine an amount of calories burned by the subject; and
a transmitting component for transmitting the heart rate, exercise, calorie, and weight information; and
an Internet-accessible website configured to receive the heart rate, exercise, calorie, and weight information.
